(19) United States
(12) Patent Application Publication
(10) Pub. No.: US 2023/0312491 A1
(43) Pub. Date: Oct. 5, 2023

(54) N-SUBSTITUTED 4-(5-PHENYL-1,3,4-OXADIAZOL-2-YL)ANILINE FOR THE TREATMENT AND PROPHYLAXIS OF HEPATITIS B VIRUS INFECTION

(71) Applicant: Hoffmann-La Roche Inc., Little Falls, NJ (US)

(72) Inventors: Xianfeng LIN, Shanghai (CN); Hongying YUN, Shanghai (CN); Bo ZHANG, Shanghai (CN); Xiufang ZHENG, Shanghai (CN)

(73) Assignee: Hoffmann-La Roche Inc., Little Falls, NJ (US)

(21) Appl. No.: 18/319,774

(22) Filed: May 18, 2023

Related U.S. Application Data
(63) Continuation of application No. PCT/EP2021/082393, filed on Nov. 22, 2021.

(30) Foreign Application Priority Data
Nov. 24, 2020 (WO) PCT/CN2020/131153

(51) Int. Cl. C07D 271/06; C07D 413/12; C07D 417/12

(52) U.S. Cl. CPC C07D 271/06; C07D 413/12; C07D 417/12

(57) ABSTRACT

The present invention provides novel compounds having the general formula (I):

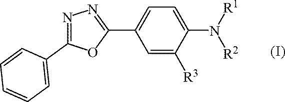

wherein R¹ to R³ are as described herein, or a pharmaceutically acceptable salt thereof, compositions including the compounds and methods of using the compounds.

### CROSS-REFERENCE TO PRIOR APPLICATIONS

This application is a continuation of International Application No. PCT/EP2021/082393 having an International Filing Date of Nov. 22, 2021 and which claims benefit under 35 U.S.C. §119 to International Application No. PCT/CN2020/131153 having an International Filing Date of Nov. 24, 2020. The entire contents of both are incorporated herein by reference.

### FIELD OF THE INVENTION

The present invention relates to N-substituted 4-(5-phenyl-1,3,4-oxadiazol-2-yl)phenyl derivatives of formula (I) useful for therapy and/or prophylaxis of HBV infection in a mammal, and in particular to HBsAg (HBV Surface antigen) and HBeAg (HBV e antigen) inhibitors as well as their manufacture and pharmaceutical compositions containing them.

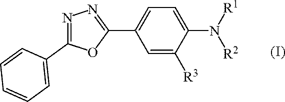

The present invention relates to compounds of formula (I) wherein R¹ to R³ are described below, or a pharmaceutically acceptable salt thereof.

### BACKGROUND OF THE INVENTION

Hepatitis B virus (HBV) is one of the most dangerous human pathogens. A safe and effective vaccine has been available for longer than two decades; however, WHO estimated that approximately 257 million people are chronically infected with HBV. Chronic Hepatitis B (CHB) infection predisposes its host to severe liver disease, including liver cirrhosis and hepatocellular carcinoma, if left untreated. HBV infection is ranked among the top unmet medical need worldwide. The currently approved drugs have contributed to substantial progress in CHB treatment; however, the cure rate remains less than 10%.

The control of viral infection needs an effective immune surveillance. Upon recognition of viral infection, the host innate immune system could respond within minutes to impede viral replication and limits the development of a chronic and persistent infection. The secretion of antiviral cytokines from infected hepatocytes and intra-hepatic immune cells is critically important for the clearance of viral infection. However, chronically infected patients only display a weak immune response due to various escape strategies adopted by the virus to counteract the host cell recognition systems and the subsequent antiviral responses.

Many observations showed that several HBV viral proteins could counteract the initial host cellular response by interfering with the viral recognition signaling system and subsequently the interferon (IFN) antiviral activity. Among these, the excessive secretion of HBV empty subviral particles (SVPs, HBsAg) may contribute to immune tolerant state observed in CHB patients. The persistent exposure to HBsAg and other viral antigens can lead to HBV-specific T-cell functional impairment and depletion (Kondo et al. Journal of Immunology (1993), 150, 4659-4671; Kondo et al. Journal of Medical Virology (2004), 74, 425-433; Fisicaro et al. Gastroenterology, (2010), 138, 682-693;). Moreover, HBsAg has been reported to suppress immune cell functions, including monocytes, dendritic cells (DCs) and natural killer (NK) cells (Op den Brouw et al. Immunology, (2009b), 126, 280-289; Woltman et al. PLoS One, (2011), 6, e15324; Shi et al. J Viral Hepat. (2012), 19, e26-33; Kondo et al. ISRN Gasteroenterology, (2013), Article ID 935295).

HBsAg is an important biomarker for prognosis and treatment response in CHB. However, the achievement of HBsAg loss and seroconversion is rarely achieved in CHB patients. HBsAg loss with or without anti-HBsAg seroconversion remains the ideal clinical treatment endpoints. Current therapies, such as nucleos(t)ide analogues, are effective in supressing HBV DNA, but are not effective in reducing HBsAg level. Nucleos(t)ide analogs, even with prolonged therapy, have demonstrated HBsAg clearance rates comparable to those observed naturally (Janssen et al. Lancet, (2005), 365, 123-129; Marcellin et al. N. Engl. J. Med., (2004), 351, 1206-1217; Buster et al. Hepatology, (2007), 46, 388-394). Therefore, there is an urgent need for the development of novel therapeutic agents that could efficiently reduce HBsAg. (Wieland, S. F. & F. V. Chisari. J Virol, (2005), 79, 9369-9380; Kumar et al. J Virol, (2011), 85, 987-995; Woltman et al. PLoS One, (2011), 6, e15324; Op den Brouw et al. Immunology, (2009b), 126, 280-289).

### SUMMARY OF THE INVENTION

Objects of the present invention are compounds of formula (I), their manufacture, medicaments based on a compound in accordance with the invention and their production as well as the use of compounds of formula (I) as HBV inhibitors and for the treatment or prophylaxis of HBV infection. The compounds of formula (I) show superior anti-HBV activity. In addition, the compounds of formula (I) also show good safety and good PK profiles.

The present invention relates to compounds of formula (I),

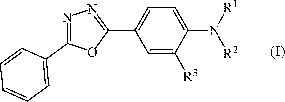

wherein
R¹ is H, C₁₋₆alkyl or —C(O)—R⁴; wherein
   R⁴ is C₁₋₆alkyl, C₁₋₆alkoxyC₁₋₆alkyl, C₃₋₇cycloalkyl, phenyl or heterocyclyl; wherein
      phenyl and heterocyclyl are unsubstituted or substituted by one or two or three substituents independently selected from halogen, C₁₋₆alkyl, C₁₋₆alkoxy, haloC₁₋ ₆alkoxy and CN;
R² is H or C₁₋₆alkyl;
R³ is H, halogen, C₁₋₆alkyl or C₁₋₆alkoxy;
wherein with the proviso that R¹, R² and R³ are not H simultaneously;
or a pharmaceutically acceptable salt thereof.

### DETAILED DESCRIPTION OF THE INVENTION

### Definitions

As used herein, the term “C₁₋₆alkyl” alone or in combination signifies a saturated, linear- or branched chain alkyl group containing 1 to 6, particularly 2 to 6 or 1 to 4 carbon atoms, for example methyl, ethyl, propyl, butyl, isobutyl, methoxyethyl, isopropyl, tert-butyl, 2,2-dimethylpropyl and the like. Particular “C₁₋₆alkyl” groups are methyl, ethyl and isopropyl.

The term “C₁₋₆alkoxy” alone or in combination signifies a group C₁₋₆alkyl—O—, wherein the “C₁₋₆alkyl” is as defined above; for example methoxy, ethoxy, propoxy, iso-propoxy, n-butoxy, iso-butoxy, 2-butoxy, tert-butoxy, pentoxy, hexyloxy and the like. Particular “C₁₋₆alkoxy” group is methoxy.

The term “C₃₋₇cycloalkyl” denotes to a saturated carbon mono or bicyclic ring or a saturated spiro- linked bicyclic carbon ring or a bridged carbon ring, containing from 3, 4, 5, 6, or 7 carbon atoms, particularly from 3 to 6 carbon atoms, for example, cyclopropyl, cyclobutyl, cyclopentyl, cyclohexyl, cycloheptyl, bicyclo[1.1.1]pentanyl and the like. Particular “C₃₋ ₇cycloalkyl” group is cyclopentyl.

The term “halogen” and “halo” are used interchangeably herein and denote fluoro, chloro, bromo, or iodo.

The term “haloC₁₋₆alkyl” denotes an alkyl group wherein at least one of the hydrogen atoms of the alkyl group is replaced by same or different halogen atoms, particularly fluoro atoms. Examples of haloC₁₋₆alkyl include monochloro-, difluoro- or trifluoro-methyl, -ethyl or -propyl, for example difluoromethyl and trifluoromethyl.

The term “haloC₁₋₆alkoxy” denotes a C₁₋₆alkoxy group wherein at least one of the hydrogen atoms of the C₁₋₆alkoxy group is replaced by same or different halogen atoms, particularly fluoro atoms. Examples of haloC₁₋₆alkoxy include monofluoro-, difluoro- or trifluoro-methoxy, -ethoxy or -propoxy, for example difluoromethoxy and trifluoromethoxy.

The term “heterocyclyl” refers to any mono-, bi-, tricyclic or spiro, saturated or unsaturated, aromatic (heteroaryl) or non-aromatic (e.g., heterocycloalkyl), ring system, having 3 to 20 ring atoms, where the ring atoms are carbon, and at least one atom in the ring or ring system is a heteroatom selected from nitrogen, sulfur or oxygen. If any ring atom of a cyclic system is a heteroatom, that system is a heterocyclyl, regardless of the point of attachment of the cyclic system to the rest of the molecule. In one example, heterocyclyl includes 3-11 ring atoms (“members”) and includes monocycles, bicycles, tricycles and spiro ring systems, wherein the ring atoms are carbon, where at least one atom in the ring or ring system is a heteroatom selected from nitrogen, sulfur or oxygen. In one example, heterocyclyl includes 3- to 7-membered monocycles having 1, 2, 3 or 4 heteroatoms selected from nitrogen, sulfur or oxygen. In another example, heterocyclyl includes 4-, 5- or 6-membered monocycles having 1, 2, 3 or 4 heteroatoms selected from nitrogen, sulfur or oxygen. In one example, heterocyclyl includes 8- to 12-membered bicycles having 1, 2, 3, 4, 5 or 6 heteroatoms selected from nitrogen, sulfur or oxygen. In another example, heterocyclyl includes 9- or 10-membered bicycles having 1, 2, 3, 4, 5 or 6 heteroatoms selected from nitrogen, sulfur or oxygen. Examplary heterocyclyls are pyridyl, pyridazinyl, oxazolyl and thiazolyl. Heterocyclyl may be optionally substituted by halogen, OH, SH, cyano, NH₂, NHCH₃, N(CH₃)₂, NO₂, N₃, C(O)CH₃, COOH, CO₂CH₃, C₁₋₆alkyl, C₁₋₆alkoxy, oxo, haloC₁₋₆alkyl, haloC₁₋₆alkoxy, phenyl or heterocyclyl.

The term “carbonyl” alone or in combination refers to the group —C(O)—.

The compounds according to the present invention may exist in the form of their pharmaceutically acceptable salts. The term “pharmaceutically acceptable salt” refers to conventional acid-addition salts or base-addition salts that retain the biological effectiveness and properties of the compounds of formula (I) and are formed from suitable non-toxic organic or inorganic acids or organic or inorganic bases. Acid-addition salts include for example those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Base-addition salts include those derived from ammonium, potassium, sodium and, quaternary ammonium hydroxides, such as for example, tetramethyl ammonium hydroxide. The chemical modification of a pharmaceutical compound into a salt is a technique well known to pharmaceutical chemists in order to obtain improved physical and chemical stability, hygroscopicity, flowability and solubility of compounds. It is for example described in Bastin R.J., et al., Organic Process Research & Development 2000, 4, 427-435. Particular are the sodium salts of the compounds of formula (I).

### HBV Inhibitors

The present invention provides (i) a compound having the general formula (I):

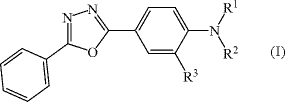

wherein
R¹ is H, C₁₋₆alkyl or —C(O)—R⁴; wherein
   R⁴ is C₁₋₆alkyl, C₁₋₆alkoxyC₁₋₆alkyl, C₃₋₇cycloalkyl, phenyl or heterocyclyl; wherein
      phenyl and heterocyclyl are unsubstituted or substituted by one or two or three substituents independently selected from halogen, C₁₋₆alkyl, C₁₋₆alkoxy, haloC₁₋ ₆alkoxy and CN;
R² is H or C₁₋₆alkyl;
R³ is H, halogen, C₁₋₆alkyl or C₁₋₆alkoxy;
wherein with the proviso that R¹, R² and R³ are not H simultaneously;
or a pharmaceutically acceptable salt thereof.

A further embodiment of the present invention is (ii) a compound of formula (I) according to (i), wherein
R¹ is H, C₁₋₆alkyl or —C(O)—R⁴; wherein
   R⁴ is C₁₋₆alkyl, C₁₋₆alkoxyC₁₋₆alkyl, C₃₋₇cycloalkyl, phenyl, pyridyl, pyridazinyl, oxazolyl or thiazolyl; wherein phenyl and pyridyl are unsubstituted or substituted by one or two or three substituents independently selected from halogen, C₁₋₆alkyl, C₁₋₆alkoxy, halogenC₁₋₆alkoxy and CN;
R² is H or C₁₋₆alkyl;
R³ is H, halogen, C₁₋₆alkyl or C₁₋₆alkoxy;
wherein with the proviso that R¹, R² and R³ are not H simultaneously;
or a pharmaceutically acceptable salt thereof.

A further embodiment of the present invention is (iii) a compound of formula (I) according to (i), wherein
R¹ is H, methyl or —C(O)—R⁴; wherein
   R⁴ is ethyl, propyl, isopropyl, butyl, isobutyl, tert-butyl, 2,2-dimethylpropyl, methoxyethyl, methoxymethyl, cyclopentyl, phenyl, pyridyl, pyridazinyl, oxazolyl or thiazolyl; wherein phenyl and pyridyl are unsubstituted or substituted by one or two or three substituents independently selected from F, Cl, methyl, methoxy, difluoromethoxy, trifluoromethoxy and CN;
R² is H or methyl;
R³ is H, F, methyl or methoxy;
wherein with the proviso that R¹, R² and R³ are not H simultaneously;
or a pharmaceutically acceptable salt thereof.

A further embodiment of the present invention is (iv) a compound of formula (I) according to (i), or a pharmaceutically acceptable salt thereof, wherein R¹ is —C(O)—R⁴; wherein R⁴ is C₁₋ ₆alkyl, C₁₋₆alkoxyC₁₋₆alkyl, C₃₋₇cycloalkyl, pyridyl, pyridazinyl or oxazolyl; wherein pyridyl is substituted one time by halogen.

A further embodiment of the present invention is (v) a compound of formula (I) according to (i), or a pharmaceutically acceptable salt thereof, wherein R⁴ is ethyl, isopropyl, methoxymethyl, cyclopentyl, pyridyl, pyridazinyl or oxazolyl; wherein pyridyl is substituted one time by Cl.

A further embodiment of the present invention is (vi) a compound of formula (I) according to (i), or a pharmaceutically acceptable salt thereof, wherein R² is H; and R³ is H.

A further embodiment of the present invention is (vii) a compound of formula (II) according to (i), or a pharmaceutically acceptable salt thereof,

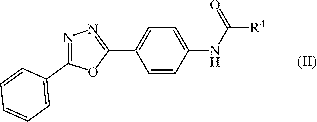

wherein R⁴ is C₁₋₆alkyl, C₁₋₆alkoxyC₁₋₆alkyl, C₃₋₇cycloalkyl, pyridyl, pyridazinyl or oxazolyl; wherein pyridyl is substituted one time by halogen.

A further embodiment of the present invention is (viii) a compound of formula (II) according to (i), or a pharmaceutically acceptable salt thereof, wherein R⁴ is ethyl, isopropyl, methoxymethyl, cyclopentyl, pyridyl, pyridazinyl or oxazolyl; wherein pyridyl is substituted one time by Cl.

In another embodiment (ix) of the present invention, particular compounds of the present invention are selected from:
N-[4-(5-phenyl-1,3,4-oxadiazol-2-yl)phenyl]propanamide;
N-[4-(5-phenyl-1,3,4-oxadiazol-2-yl)phenyl]butanamide;
N-[4-(5-phenyl-1,3,4-oxadiazol-2-yl)phenyl]pentanamide;
3-methyl-N-[4-(5-phenyl-1,3,4-oxadiazol-2-yl)phenyl]butanamide;
3-methoxy-N-[4-(5-phenyl-1,3,4-oxadiazol-2-yl)phenyl]propanamide;
2-methyl-N-[4-(5-phenyl-1,3,4-oxadiazol-2-yl)phenyl]propanamide;
N-[4-(5-phenyl-1,3,4-oxadiazol-2-yl)phenyl]cyclopentanecarboxamide;
2-fluoro-N-[4-(5-phenyl-1,3,4-oxadiazol-2-yl)phenyl]benzamide;
2-chloro-N-[4-(5-phenyl-1,3,4-oxadiazol-2-yl)phenyl]benzamide;
3-fluoro-5-methyl-N-[4-(5-phenyl-1,3,4-oxadiazol-2-yl)phenyl]benzamide;
3-chloro-5-methyl-N-[4-(5-phenyl-1,3,4-oxadiazol-2-yl)phenyl]benzamide;
4-fluoro-N-[4-(5-phenyl-1,3,4-oxadiazol-2-yl)phenyl]benzamide;
4-chloro-N-[4-(5-phenyl-1,3,4-oxadiazol-2-yl)phenyl]benzamide;
N-[4-(5-phenyl-1,3,4-oxadiazol-2-yl)phenyl]-2-(trifluoromethoxy)benzamide;
2-(difluoromethoxy)-N-[4-(5-phenyl-1,3,4-oxadiazol-2-yl)phenyl]benzamide;
N-[4-(5-phenyl-1,3,4-oxadiazol-2-yl)phenyl]-3-(trifluoromethoxy)benzamide;
3-(difluoromethoxy)-N-[4-(5-phenyl-1,3,4-oxadiazol-2-yl)phenyl]benzamide;
4-cyano-N-[4-(5-phenyl-1,3,4-oxadiazol-2-yl)phenyl]benzamide;
N-[4-(5-phenyl-1,3,4-oxadiazol-2-yl)phenyl]pyridine-4-carboxamide;
2-chloro-N-[4-(5-phenyl-1,3,4-oxadiazol-2-yl)phenyl]pyridine-4-carboxamide;
2-methoxy-N-[4-(5-phenyl-1,3,4-oxadiazol-2-yl)phenyl]pyridine-4-carboxamide;
6-chloro-N-[4-(5-phenyl-1,3,4-oxadiazol-2-yl)phenyl]pyridine-2-carboxamide;
5-chloro-N-[4-(5-phenyl-1,3,4-oxadiazol-2-yl)phenyl]pyridine-2-carboxamide;
N-[4-(5-phenyl-1,3,4-oxadiazol-2-yl)phenyl]pyridazine-4-carboxamide;
N-[4-(5-phenyl-1,3,4-oxadiazol-2-yl)phenyl]pyridazine-3-carboxamide;
N-[4-(5-phenyl-1,3,4-oxadiazol-2-yl)phenyl]oxazole-2-carboxamide;
N-[4-(5-phenyl-1,3,4-oxadiazol-2-yl)phenyl]thiazole-2-carboxamide;
2,2-dimethyl-N-[4-(5-phenyl-1,3,4-oxadiazol-2-yl)phenyl]propanamide;
3,3-dimethyl-N-[4-(5-phenyl-1,3,4-oxadiazol-2-yl)phenyl]butanamide;
2-methyl-4-(5-phenyl-1,3,4-oxadiazol-2-yl)aniline;
2-methoxy-4-(5-phenyl-1,3,4-oxadiazol-2-yl)aniline;
2-fluoro-4-(5-phenyl-1,3,4-oxadiazol-2-yl)aniline; and
N,N-dimethyl-4-(5-phenyl-1,3,4-oxadiazol-2-yl)aniline;
or a pharmaceutically acceptable salt thereof.

In another embodiment (x) of the present invention, particular compounds of the present invention are selected from:
N-[4-(5-phenyl-1,3,4-oxadiazol-2-yl)phenyl]propanamide;
3-methoxy-N-[4-(5-phenyl-1,3,4-oxadiazol-2-yl)phenyl]propanamide;
2-methyl-N-[4-(5-phenyl-1,3,4-oxadiazol-2-yl)phenyl]propanamide;
N-[4-(5-phenyl-1,3,4-oxadiazol-2-yl)phenyl]cyclopentanecarboxamide;
6-chloro-N-[4-(5-phenyl-1,3,4-oxadiazol-2-yl)phenyl]pyridine-2-carboxamide;
N-[4-(5-phenyl-1,3,4-oxadiazol-2-yl)phenyl]pyridazine-3-carboxamide; and
N-[4-(5-phenyl-1,3,4-oxadiazol-2-yl)phenyl]oxazole-2-carboxamide;
or a pharmaceutically acceptable salt thereof.

### Synthesis

The compounds of the present invention can be prepared by any conventional means. Suitable processes for synthesizing these compounds as well as their starting materials are provided in the schemes below and in the examples. All substituents, in particular, R¹ to R⁴, are as defined above unless otherwise indicated. Furthermore, and unless explicitly otherwise stated, all reactions, reaction conditions, abbreviations and symbols have the meanings well known to a person of ordinary skill in organic chemistry.

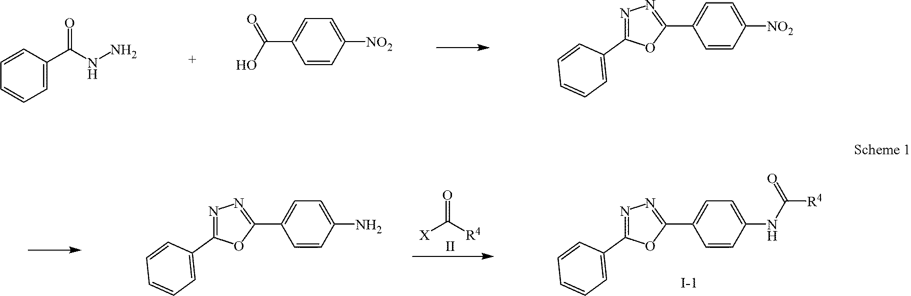

Wherein X is halogen or OH.

A compound of formula 1-1 can be prepared according to Scheme 1. 2-(4-Nitrophenyl)-5-phenyl-1,3,4-oxadiazole can be prepared by coupling of benzohydrazide and 4-nitrobenzoic acid with a suitable coupling reagent, such as O-(7-azabenzotriazol-1-yl)-N,N,N′,N′-tetramethyluronium hexafluorophosphate and a suitable base, such as N,N-diisopropylethylamine in a suitable solvent, such as acetonitrile, followed by cyclization in the presence of p-toluenesulfonyl chloride. 2-(4-Nitrophenyl)-5-phenyl-1,3,4-oxadiazole can be reduced by a reducing agent, such as palladium on carbon in the presence of hydrazine hydrate in a suitable solvent, such ethanol to give 4-(5-phenyl-1,3,4-oxadiazol-2-yl)aniline. The compound of formula 1-1 can be prepared from coupling of 4-(5-phenyl-1,3,4-oxadiazol-2-yl)aniline with a compound of formula II in the presence of a suitable coupling reagent, such as propylphosphonic anhydride and O-(7-azabenzotriazol-1-yl)-N,N,N′,N′-tetramethyluronium hexafluorophosphate, and a suitable base, such as triethylamine.

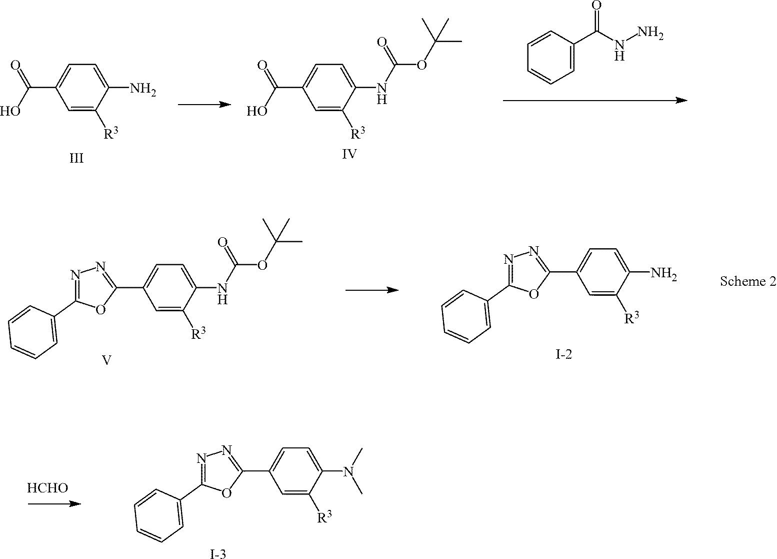

Compounds of formula 1-2 and formula 1-3 can be prepared according to Scheme 2. A compound of formula III can be protected by di-t-butyldicarbonate in the presence of a suitable base, such as sodium hydroxide to afford a compound of formula IV. Coupling of benzohydrazide and the compound of formula IV with a suitable coupling reagent, such as O-(7-azabenzotriazol-1-yl)-N,N,N′,N′-tetramethyluronium hexafluorophosphate and a suitable base, such as N,N-diisopropylethylamine in a suitable solvent, such as acetonitrile, followed by cyclization in the presence of p-toluenesulfonyl chloride to afford a compound of formula V. The compound of formula V can be deprotected with a suitable acid, such as HCl in dioxane to afford a compound of formula 1-2. The compound of formula 1-3 can be prepared from a compound of formula 1-2 by reductive amination reaction with formaldehyde in the presence of sodium cyanoborohydride.

This invention also relates to a process for the preparation of a compound of formula (I) comprising at least one of the following steps:
(a) coupling of a compound of formula (II),

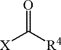

(II), with 4-(5-phenyl-1,3,4-oxadiazol-2-yl)aniline in the presence of a coupling reagent and a base;
(b) deprotection of a compound of formula (V),

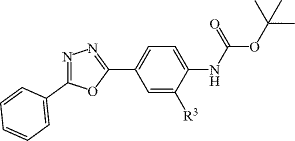

(V), in the presence of an acid;
(c) reductive amination of a compound of formula (I-2),

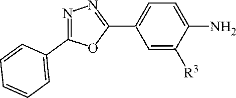

(I-2), with formaldehyde in the presence of sodium cyanoborohydride;
wherein R¹ to R⁴ are defined above; X is halogen or OH.
The coupling reagent in step (a) can be for example propylphosphonic anhydride or O-(7-azabenzotriazol-1-yl)-N,N,N′,N′-tetramethyluronium hexafluorophosphate;
The base in step (a) can be for example triethylamine;
The acid in step (b) can be for example HCl;

A compound of formula (I) or (II) when manufactured according to the above process is also an object of the invention.

The compound of this invention also shows good safety and PK profile.

### PHARMACEUTICAL COMPOSITIONS AND ADMINISTRATION

The invention also relates to a compound of formula (I) for use as therapeutically active substance. Another embodiment provides pharmaceutical compositions or medicaments containing the compounds of the invention and a therapeutically inert carrier, diluent or excipient, as well as methods of using the compounds of the invention to prepare such compositions and medicaments. In one example, compounds of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) is formulated in an acetate buffer, at pH 5. In another embodiment, the compounds of formula (I) are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to reduction of HBsAg and HBeAg in HBV patients. For example, such amount may be below the amount that is toxic to normal cells, or the mammal as a whole.

In one example, the pharmaceutically effective amount of the compound of the invention administered parenterally per dose will be in the range of about 0.1 to 100 mg/kg, alternatively about 0.1 to 50 mg/kg of patient body weight per day, with the typical initial range of compound used being 0.3 to 15 mg/kg/day. In another embodiment, oral unit dosage forms, such as tablets and capsules, preferably contain from about 25 to about 1000 mg of the compound of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel’s Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 25 to 500 mg of the compound of the invention compounded with about 90 to 30 mg anhydrous lactose, about 5 to 40 mg sodium croscarmellose, about 5 to 30 mg polyvinylpyrrolidone (PVP) K30, and about 1 to 10 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 5 to 400 mg, of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

An embodiment, therefore, includes a pharmaceutical composition comprising a compound of formula (I), or a pharmaceutically acceptable salt thereof.

In a further embodiment includes a pharmaceutical composition comprising a compound of formula (I), or a pharmaceutically acceptable salt thereof, together with a pharmaceutically acceptable carrier or excipient.

Another embodiment includes a pharmaceutical composition comprising a compound of formula (I), or a pharmaceutically acceptable salt thereof for use in the treatment of HBV infection.

### INDICATIONS AND METHODS OF TREATMENT

The compounds of the invention have anti-HBV activity. Accordingly, the compounds of the invention are useful for the treatment or prophylaxis of HBV infection.

The invention also relates to the use of a compound of formula (I) for the inhibition of HBeAg.

The invention further relates to the use of a compound of formula (I) for the inhibition of HBsAg.

The invention relates to the use of a compound of formula (I) for the inhibition of HBV DNA.

The invention relates to the use of a compound of formula (I) for the treatment or prophylaxis of HBV infection.

The use of a compound of formula (I) for the preparation of medicaments useful in the treatment or prophylaxis diseases that are related to HBV infection is an object of the invention.

The invention relates in particular to the use of a compound of formula (I) for the preparation of a medicament for the treatment or prophylaxis of HBV infection.

Another embodiment includes a method for the treatment or prophylaxis of HBV infection, which method comprises administering an effective amount of a compound of formula (I), or a pharmaceutically acceptable salt thereof.

### EXAMPLES

The invention will be more fully understood by reference to the following examples. They should not, however, be construed as limiting the scope of the invention.

Abbreviations used herein are as follows:

| | |
|---|---|
| DMSO-d₆: | deuterated dimethylsulfoxide |
| HPLC: | high performance liquid chromatography |
| h: | hour |
| IC₅₀: | the half maximal inhibitory concentration |
| LC/MS: | liquid chromatography/mass spectrometry |
| MHz: | megahertz |
| mL: | milliliter |
| mmol: | millimole |
| MS (ESI): | mass spectroscopy (electron spray ionization) |
| NMR: | nuclear magnetic resonance |
| obsd. | observed |
| δ: | chemical shift |

### General Experimental Conditions

Intermediates and final compounds were purified by flash chromatography using one of the following instruments: i) Biotage SP1 system and the Quad 12/25 Cartridge module. ii) ISCO combi-flash chromatography instrument. Silica gel Brand and pore size: i) KP-SIL 60 Å, particle size: 40-60 µm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using X Bridge™ Perp C₁₈ (5 µm, OBD™ 30 × 100 mm) column or SunFire™ Perp C₁₈ (5 µm, OBD™ 30 × 100 mm) column.

LC/MS spectra were obtained using an Acquity Ultra Performance LC - 3100 Mass Detector or Acquity Ultra Performance LC - SQ Detector. Standard LC/MS conditions were as follows (running time 3 minutes):
Acidic condition: A: 0.1% formic acid in H₂O; B: 0.1% formic acid in acetonitrile;
Basic condition: A: 0.05% NH₃·H₂O in H₂O; B: acetonitrile;
Neutral condition: A: H₂O; B: acetonitrile.

Mass spectra (MS): generally only ions which indicate the parent mass are reported, and unless otherwise stated the mass ion quoted is the positive mass ion (M+H)⁺.

The microwave assisted reactions were carried out in a Biotage Initiator Sixty or CEM Discover.

NMR Spectra were obtained using Bruker Avance 400 MHz.

All reactions involving air-sensitive reagents were performed under an argon atmosphere. Reagents were used as received from commercial suppliers without further purification unless otherwise noted.

### Preparative Examples

The invention will be more fully understood by reference to the following examples. They should not, however, be construed as limiting the scope of the invention.

### Example 1

### N-(5-phenyl-1,3,4-oxadiazol-2-yl)phenyl]propanamide

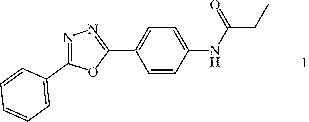 [SMILES: CCC(=O)Nc1ccc(-c2nnc(-c3ccccc3)o2)cc1]

The title compound was prepared according to the following scheme:

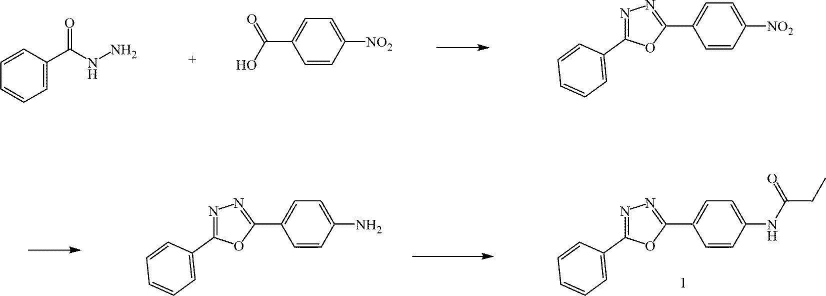

### Step 1: Preparation of 2-(4-nitrophenyl)-5-phenyl-1,3,4-oxadiazole

A mixture of benzohydrazide (3.26 g, 23.9 mmol), 4-nitrobenzoic acid (4.0 g, 23.9 mmol), O-(7-azabenzotriazol-1-yl)-N,N,N′,N′-tetramethyluronium hexafluorophosphate (10.01 g, 26.3 mmol) and N,N-diisopropylethylamine (9.28 g, 71.8 mmol) in acetonitrile (40 mL) was stirred at 25° C. for 12 h. After p-toluenesulfonyl chloride (13.69 g, 71.8 mmol) was added, the mixture was stirred for another 3 h. The resulting mixture was treated with ammonium hydroxide solution and filtered. The filter cake was washed with acetonitrile to give 2-(4-nitrophenyl)-5-phenyl-1,3,4-oxadiazole (4.5 g) as a white solid. MS obsd. (ESI⁺) [(M+H)⁺]: 268.1.

### Step 2: Preparation of 4-(5-phenyl-1,3,4-oxadiazol-2-yl)aniline

To a mixture of 2-(4-nitrophenyl)-5-phenyl-1,3,4-oxadiazole (5.3 g, 19.8 mmol) and hydrazine hydrate (5.84 g, 99.2 mmol) in ethanol (100 mL) was added 10% palladium on carbon (100 mg). After being heated with stirring at 80° C. for 5 h, the resulting mixture was filtered by silica gel immediately. The product was precipitated from filtrate after the temperature cooled down, which was collected by filtration to give 4-(5-phenyl-1,3,4-oxadiazol-2-yl)aniline (2.5 g) as a light yellow solid. MS obsd. (ESI⁺) [(M+H)⁺]: 238.1.

### Step 3: Preparation of N-[4-(5-phenyl-1,3,4-oxadiazol-2-yl)phenyl]propanamide (Example 1)

To a solution of 4-(5-phenyl-1,3,4-oxadiazol-2-yl)aniline (70.0 mg, 0.3 mmol) in dichloromethane (4 mL) was added triethylamine (0.12 mL, 0.9 mmol) and propionyl chloride (54.59 mg, 0.6 mmol). After being stirred at 20° C. for 5 h, the mixture was concentrated. The crude product was purified by preparative HPLC to give N-[4-(5-phenyl-1,3,4-oxadiazol-2-yl)phenyl]propanamide (Example 1, 30 mg) as a white solid. MS obsd. (ESI⁺) [(M+H)⁺]: 294.1. ¹H NMR (400 MHz, DMSO-d₆) δ ppm: 10.27 (s, 1H), 8.16 - 8.11 (m, 2H), 8.08 (d, J= 8.8 Hz, 2H), 7.86 (d, J= 8.8 Hz, 2H), 7.68 - 7.61 (m, 3H), 2.39 (q, J= 7.5 Hz, 2H), 1.11 (t, J= 7.5 Hz, 3H).

### Example 2

### N-(5-phenyl-1,3,4-oxadiazol-2-yl)phenyl]butanamide

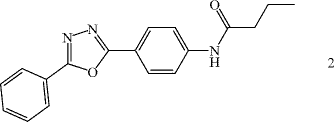 [SMILES: CCCC(=O)Nc1ccc(-c2nnc(-c3ccccc3)o2)cc1]

The title compound was prepared in analogy to the procedure described for the preparation of Example 1, by using butyryl chloride instead of propionyl chloride. The product was purified by preparative HPLC to afford Example 2 as a white solid. MS obsd. (ESI⁺) [(M+H)⁺]: 308.1. ¹H NMR (400 MHz, DMSO-d₆) δ ppm: 10.27 (s, 1H), 8.19 - 8.04 (m, 4H), 7.86 (d, J= 8.8 Hz, 2H), 7.70 - 7.60 (m, 3H), 2.36 - 2.31 (m, 2H), 1.71 - 1.60 (m, 2H), 0.94 (t, J= 7.4 Hz, 3H).

### Example 3

### N-(5-phenyl-1,3,4-oxadiazol-2-yl)phenyl]pentanamide

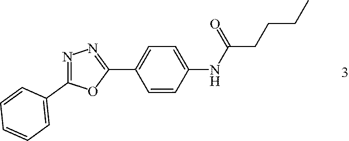 [SMILES: CCCCC(=O)Nc1ccc(-c2nnc(-c3ccccc3)o2)cc1]

The title compound was prepared in analogy to the procedure described for the preparation of Example 1, by using valeryl chloride instead of propionyl chloride. The product was purified by preparative HPLC to afford Example 3 as a white solid. MS obsd. (ESI⁺) [(M+H)⁺]: 322.1. ¹H NMR (400 MHz, DMSO-d₆) δ ppm: 10.27 (s, 1H), 8.16 - 8.11 (m, 2H), 8.08 (d, J= 8.8 Hz, 2H), 7.85 (d, J= 8.8 Hz, 2H), 7.69 - 7.61 (m, 3H), 2.37 (t, J= 7.5 Hz, 2H), 1.65 - 1.50 (m, 2H), 1.39 - 1.30 (m, 2H), 0.92 (t, J= 7.3 Hz, 3H).

### Example 4

### 3-methyl-N-[4-(5-phenyl-1,3,4-oxadiazol-2-yl)phenyl]butanamide

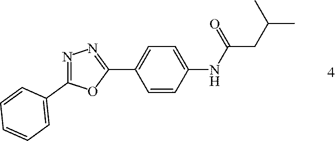 [SMILES: CC(C)CC(=O)Nc1ccc(-c2nnc(-c3ccccc3)o2)cc1]

The title compound was prepared in analogy to the procedure described for the preparation of Example 1, by using isovaleryl chloride instead of propionyl chloride. The product was purified by preparative HPLC to afford Example 4 as a white solid. MS obsd. (ESI⁺) [(M+H)⁺]: 322.2. ¹H NMR (400 MHz, DMSO-d₆) δ ppm: 10.26 (s, 1H), 8.17 - 8.11 (m, 2H), 8.08 (d,J= 8.9 Hz, 2H), 7.86 (d, J= 8.9 Hz, 2H), 7.68 - 7.61 (m, 3H), 2.25 (d, J= 7.2 Hz, 2H), 2.15 - 2.07 (m, 1 H), 0.96 (d, J= 6.7 Hz, 6H).

### Example 5

### 3-methoxy-N-[4-(5-phenyl-1,3,4-oxadiazol-2-yl)phenyl]propanamide

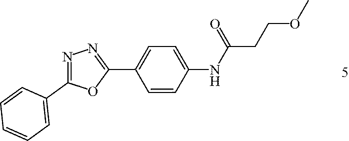 [SMILES: COCCC(=O)Nc1ccc(-c2nnc(-c3ccccc3)o2)cc1]

The title compound was prepared in analogy to the procedure described for the preparation of Example 1, by using 3-methoxypropanoyl chloride instead of propionyl chloride. The product was purified by preparative HPLC to afford Example 5 as a white solid. MS obsd. (ESI⁺) [(M+H)⁺]: 324.1. ¹H NMR (400 MHz, DMSO-d₆) δ ppm: 10.34 (s, 1H), 8.20 - 8.04 (m, 4H), 7.86 (d, J=8.8 Hz, 2H), 7.71 - 7.60 (m, 3H), 3.65 (t, J= 6.1 Hz, 2H), 3.26 (s, 3H), 2.61 (t, J= 6.1 Hz, 2H).

### Example 6

### 2-methyl-N-[4-(5-phenyl-1,3,4-oxadiazol-2-yl)phenyl]propanamide

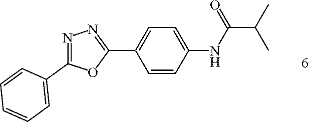 [SMILES: CC(C)C(=O)Nc1ccc(-c2nnc(-c3ccccc3)o2)cc1]

The title compound was prepared in analogy to the procedure described for the preparation of Example 1, by using isobutyryl chloride instead of propionyl chloride. The product was purified by preparative HPLC to afford Example 6 as a white solid. MS obsd. (ESI⁺) [(M+H)⁺]: 308.1. ¹H NMR (400 MHz, DMSO-d₆) δ ppm: 10.23 (s, 1H), 8.16 - 8.12 (m, 2H), 8.08 (d, J= 8.8 Hz, 2H), 7.88 (d, J= 8.8 Hz, 2H), 7.67 - 7.62 (m, 3H), 2.65 - 2.61 (m, 1H), 1.15 (s, 3H), 1.13 (s, 3H).

### Example 7

### N-(5-phenyl-l,3,4-oxadiazol-2-yl)phenyl]cyclopentanecarboxamide

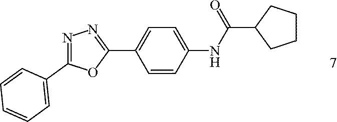 [SMILES: O=C(Nc1ccc(-c2nnc(-c3ccccc3)o2)cc1)C1CCCC1]

The title compound was prepared in analogy to the procedure described for the preparation of Example 1, by using cyclopentanecarbonyl chloride instead of propionyl chloride. The product was purified by preparative HPLC to afford Example 7 as a white solid. MS obsd. (ESI⁺) [(M+H)⁺]: 334.1. ¹H NMR (400 MHz, DMSO-d₆) δ ppm: 10.27 (s, 1H), 8.17 - 8.11 (m, 2H), 8.08 (d, J= 8.8 Hz, 2H), 7.87 (d, J= 8.8 Hz, 2H), 7.69 - 7.60 (m, 3H), 2.88 - 2.79 (m, 1H), 1.95 - 1.83 (m, 2H), 1.81 - 1.65 (m, 4H), 1.63 - 1.51 (m, 2H).

### Example 8

### 2-fluoro-N-[4-(5-phenyl-1,3,4-oxadiazol-2-yl)phenyl]benzamide

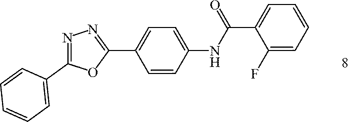 [SMILES: O=C(Nc1ccc(-c2nnc(-c3ccccc3)o2)cc1)c1ccccc1F]

The title compound was prepared in analogy to the procedure described for the preparation of Example 1, by using cyclopentanecarbonyl chloride instead of propionyl chloride. The product was purified by preparative HPLC to afford Example 8 as a white solid. MS obsd. (ESI⁺) [(M+H)⁺]: 360.1. ¹H NMR (400 MHz, DMSO-d₆) δ ppm: 10.83 (s, 1H), 8.23 - 8.09 (m, 4H), 8.00 (d, J= 8.8 Hz, 2H), 7.72 (dt, J= 1.6, 7.4 Hz, 1H), 7.69 - 7.59 (m, 4H), 7.44 - 7.34 (m, 2H).

### Example 9

### 2-chloro-N-[4-(5-phenyl-1,3,4-oxadiazol-2-yl)phenyl]benzamide

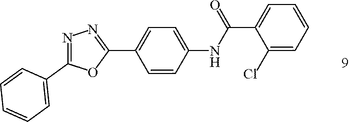 [SMILES: O=C(Nc1ccc(-c2nnc(-c3ccccc3)o2)cc1)c1ccccc1Cl]

The title compound was prepared in analogy to the procedure described for the preparation of Example 1, by using 2-chlorobenzoyl chloride instead of propionyl chloride. The product was purified by preparative HPLC to afford Example 9 as a white solid. MS obsd. (ESI⁺) [(M+H)⁺]: 376.1. ¹H NMR (400 MHz, DMSO-d₆) δ ppm: 10.91 (s, 1H), 8.19 - 8.11 (m, 4H), 7.99 (d, J= 8.8 Hz, 2H), 7.69 - 7.46 (m, 7H).

### Example 10

### 2-chloro-N-[4-(5-phenyl-1,3,4-oxadiazol-2-yl)phenyl]benzamide

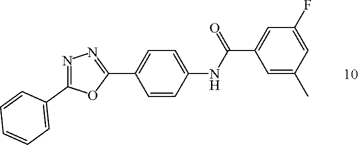 [SMILES: Cc1cc(F)cc(C(=O)Nc2ccc(-c3nnc(-c4ccccc4)o3)cc2)c1]

The title compound was prepared in analogy to the procedure described for the preparation of Example 1, by using 3-fluoro-5-methyl-benzoyl chloride instead of propionyl chloride. The product was purified by preparative HPLC to afford Example 10 as a white solid. MS obsd. (ESI⁺) [(M+H)⁺]: 374.0. ¹H NMR (400 MHz, DMSO-d₆) δ ppm: 10.62 (s, 1H), 8.19 - 8.11 (m, 4H), 8.09 - 8.02 (m, 2H), 7.69 - 7.62 (m, 4H), 7.61 - 7.57 (m, 1H), 7.36 - 7.29 (m, 1H), 2.43 (s, 3H).

### Example 11

### 3-chloro-5-methyl-N-[4-(5-phenyl-1,3,4-oxadiazol-2-yl)phenyl]benzamide

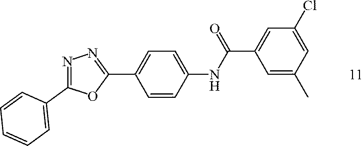 [SMILES: Cc1cc(Cl)cc(C(=O)Nc2ccc(-c3nnc(-c4ccccc4)o3)cc2)c1]

The title compound was prepared in analogy to the procedure described for the preparation of Example 1, by using 3-chloro-5-methylbenzoic acid and propylphosphonic anhydride instead of propionyl chloride. The product was purified by preparative HPLC to afford Example 11 as a light yellow solid. MS obsd. (ESI⁺) [(M+H)⁺]: 390.0. ¹H NMR (400 MHz, DMSO-d₆) δ ppm: 10.65 (s, 1H), 8.19 - 8.11 (m, 4H), 8.06 (d, J= 8.9 Hz, 2H), 7.88 - 7.76 (m, 2H), 7.70 - 7.61 (m, 3H), 7.55 (s, 1H), 2.43 (s, 3H).

### Example 12

### 4-fluoro-N-[4-(5-phenyl-1,3,4-oxadiazol-2-yl)phenyl]benzamide

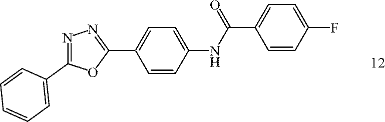 [SMILES: O=C(Nc1ccc(-c2nnc(-c3ccccc3)o2)cc1)c1ccc(F)cc1]

The title compound was prepared in analogy to the procedure described for the preparation of Example 1, by using 4-fluorobenzoic acid and propylphosphonic anhydride instead of propionyl chloride. The product was purified by preparative HPLC to afford Example 12 as a white solid. MS obsd. (ESI⁺) [(M+H)⁺]: 360.1. ¹H NMR (400 MHz, DMSO-d₆) δ ppm: 10.62 (s, 1H), 8.23 - 8.02 (m, 8H), 7.71 - 7.60 (m, 3H), 7.41 (t, J= 8.8 Hz, 2H).

### Example 13

### 4-chloro-N-[4-(5-phenyl-1,3,4-oxadiazol-2-yl)phenyl]benzamide

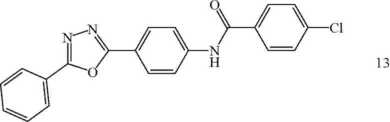 [SMILES: O=C(Nc1ccc(-c2nnc(-c3ccccc3)o2)cc1)c1ccc(Cl)cc1]

The title compound was prepared in analogy to the procedure described for the preparation of Example 1, by using 4-chlorobenzoyl chloride instead of propionyl chloride. The product was purified by preparative HPLC to afford Example 13 as a white solid. MS obsd. (ESI⁺) [(M+H)⁺]: 376.0. ¹H NMR (400 MHz, DMSO-d₆) δ ppm: 10.68 (s, 1H), 8.20 - 8.13 (m, 4H), 8.10 - 8.00 (m, 4H), 7.70 - 7.61 (m, 5H).

### Example 14

### N-(5-phenyl-1,3,4-oxadiazol-2-yl)phenyl]-2-(trifluoromethoxy)benzamide

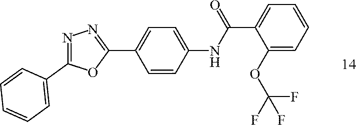 [SMILES: O=C(Nc1ccc(-c2nnc(-c3ccccc3)o2)cc1)c1ccccc1OC(F)(F)F]

The title compound was prepared in analogy to the procedure described for the preparation of Example 1, by using 2-(trifluoromethoxy)benzoyl chloride instead of propionyl chloride. The product was purified by preparative HPLC to afford Example 14 as a light yellow solid. MS obsd. (ESI⁺) [(M+H)⁺]: 426.1. ¹H NMR (400 MHz, DMSO-d₆) δ ppm: 10.91 (s, 1H), 8.21 - 8.10 (m, 4H), 7.97 (d, J= 8.7 Hz, 2H), 7.77 (dd, J= 1.6, 7.6 Hz, 1H), 7.73 - 7.62 (m, 4H), 7.60 - 7.51 (m, 2H).

### Example 15

### 2-(difluoromethoxy)-N-[4-(5-phenyl-1,3,4-oxadiazol-2-yl)phenyl]benzamide

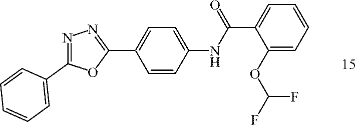 [SMILES: O=C(Nc1ccc(-c2nnc(-c3ccccc3)o2)cc1)c1ccccc1OC(F)F]

The title compound was prepared in analogy to the procedure described for the preparation of Example 1, by using 3-(difluoromethoxy)benzoic acid and O-(7-azabenzotriazol-1-yl)-N,N,N′,N′-tetramethyluronium hexafluorophosphate instead of propionyl chloride. The product was purified by preparative HPLC to afford Example 15 as a light yellow solid. MS obsd. (ESI⁺) [(M+H)⁺]: 408.1. ¹H NMR (400 MHz, DMSO-d₆) δ ppm: 10.76 (s, 1H), 8.20 - 8.09 (m, 3H), 8.20 - 8.09 (m, 1H), 7.97 (d, J= 8.7 Hz, 2H), 7.73 - 7.56 (m, 5H), 7.41 (d, J= 8.0 Hz, 1H), 7.33 (d, J=8.0 Hz, 1H), 7.24 (t,J= 73.6 Hz, 1H).

### Example 16

### N-(5-phenyl-1,3,4-oxadiazol-2-yl)phenyl]-3-(trifluoromethoxy)benzamide

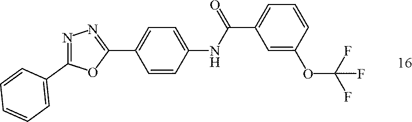 [SMILES: O=C(Nc1ccc(-c2nnc(-c3ccccc3)o2)cc1)c1cccc(OC(F)(F)F)c1]

The title compound was prepared in analogy to the procedure described for the preparation of Example 1, by using 3-(trifluoromethoxy)benzoyl chloride instead of propionyl chloride. The product was purified by preparative HPLC to afford Example 16 as a light yellow solid. MS obsd. (ESI⁺) [(M+H)⁺]: 426.0. ¹H NMR (400 MHz, DMSO-d₆) δ ppm: 10.74 (s, 1H), 8.23 - 8.12 (m, 4H), 8.10 - 8.03 (m, 3H), 7.96 (s, 1H), 7.77 - 7.61 (m, 5H).

### Example 17

### 3-(difluoromethoxy)-N-[4-(5-phenyl-1,3,4-oxadiazol-2-yl)phenyl]benzamide

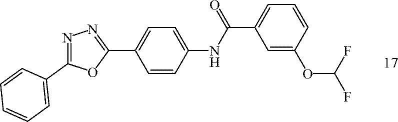 [SMILES: O=C(Nc1ccc(-c2nnc(-c3ccccc3)o2)cc1)c1cccc(OC(F)F)c1]

The title compound was prepared in analogy to the procedure described for the preparation of Example 1, by using 3-(difluoromethoxy)benzoic acid and O-(7-azabenzotriazol-1-yl)-N,N,N′,N′-tetramethyluronium hexafluorophosphate instead of propionyl chloride. The product was purified by preparative HPLC to afford Example 17 as a light yellow solid. MS obsd. (ESI⁺) [(M+H)⁺]: 408.1. ¹H NMR (400 MHz, DMSO-d₆) δ ppm: 10.68 (s, 1H), 8.21 - 8.12 (m, 4H), 8.09 - 8.03 (m, 2H), 7.89 (d, J= 7.8 Hz, 1H), 7.78 (s, 1H), 7.69 - 7.60 (m, 4H), 7.55 (s, 1H), 7.45 (dd, J= 2.2, 8.2 Hz, 1H), 7.36 (m, 1H).

### Example 18

### 4-cyano-N-[4-(5-phenyl-1,3,4-oxadiazol-2-yl)phenyl]benzamide

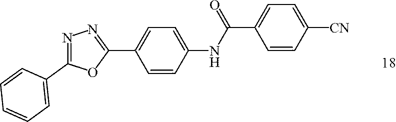 [SMILES: N#Cc1ccc(C(=O)Nc2ccc(-c3nnc(-c4ccccc4)o3)cc2)cc1]

The title compound was prepared in analogy to the procedure described for the preparation of Example 1, by using 4-cyanobenzoic acid and propylphosphonic anhydride instead of propionyl chloride. The product was purified by preparative HPLC to afford Example 18 as a white solid. MS obsd. (ESI⁺) [(M+H)⁺]: 367.1. ¹H NMR (400 MHz, DMSO-d₆) δ ppm: 10.84 (s, 1H), 8.23 - 8.12 (m, 6H), 8.10 - 8.03 (m, 4H), 7.73 - 7.60 (m, 3H).

### Example 19

### N-(5-phenyl-1,3,4-oxadiazol-2-yl)phenyl]pyridine-4-carboxamide

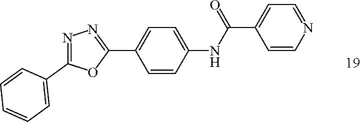 [SMILES: O=C(Nc1ccc(-c2nnc(-c3ccccc3)o2)cc1)c1ccncc1]

The title compound was prepared in analogy to the procedure described for the preparation of Example 1, by using isonicotinic acid and propylphosphonic anhydride instead of propionyl chloride. The product was purified by preparative HPLC to afford Example 19 as a light yellow solid. MS obsd. (ESI⁺) [(M+H)⁺]: 343.0. ¹H NMR (400 MHz, DMSO-d₆) δ ppm: 10.87 (s, 1H), 8.88 - 8.79 (m, 2H), 8.23 - 8.12 (m, 4H), 8.10 - 8.03 (m, 2H), 7.95 - 7.87 (m, 2H), 7.72 - 7.60 (m, 3H).

### Example 20

### 2-chloro-N-[4-(5-phenyl-1,3,4-oxadiazol-2-yl)phenyl]pyridine-4-carboxamide

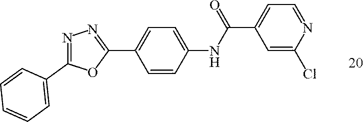 [SMILES: O=C(Nc1ccc(-c2nnc(-c3ccccc3)o2)cc1)c1ccnc(Cl)c1]

The title compound was prepared in analogy to the procedure described for the preparation of Example 1, by using 2-chloroisonicotinic acid and propylphosphonic anhydride instead of propionyl chloride. The product was purified by preparative HPLC to afford Example 20 as a white solid. MS obsd. (ESI⁺) [(M+H)⁺]: 377.2. ¹H NMR (400 MHz, DMSO-d₆) δ ppm: 10.93 (s, 1H), 8.66 (d, J= 5.0 Hz, 1H), 8.21 - 8.11 (m, 4H), 8.07 - 8.02 (m, 3H), 7.91 (dd, J= 1.4, 5.0 Hz, 1H), 7.69 - 7.62 (m, 3H).

### Example 21

### 2-methoxy-N-[4-(5-phenyl-1,3,4-oxadiazol-2-yl)phenyl]pyridine-4-carboxamide

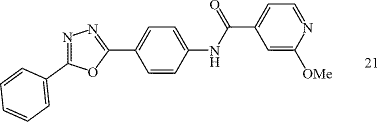 [SMILES: COc1cc(C(=O)Nc2ccc(-c3nnc(-c4ccccc4)o3)cc2)ccn1]

The title compound was prepared in analogy to the procedure described for the preparation of Example 1, by using 2-methoxyisonicotinic acid and propylphosphonic anhydride instead of propionyl chloride. The product was purified by preparative HPLC to afford Example 21 as a white solid. MS obsd. (ESI⁺) [(M+H)⁺]: 373.1. ¹H NMR (400 MHz, DMSO-d₆) δ ppm: 10.78 (s, 1H), 8.38 (d, J= 5.3 Hz, 1H), 8.19 - 8.10 (m, 4H), 8.06 - 8.04 (m, 2H), 7.70 - 7.60 (m, 3H), 7.47 (d, J= 5.3 Hz, 1H), 7.35 (s, 1H), 3.94 (s, 3H).

### Example 22

### 6-chloro-N-[4-(5-phenyl-1,3,4-oxadiazol-2-yl)phenyl]pyridine-2-carboxamide

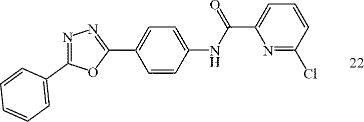 [SMILES: O=C(Nc1ccc(-c2nnc(-c3ccccc3)o2)cc1)c1cccc(Cl)n1]

The title compound was prepared in analogy to the procedure described for the preparation of Example 1, by using 6-chloropyridine-2-carboxylic acid and propylphosphonic anhydride instead of propionyl chloride. The product was purified by preparative HPLC to afford Example 22 as a white solid. MS obsd. (ESI⁺) [(M+H)⁺]: 377.0. ¹H NMR (400 MHz, DMSO-d₆) δ ppm: 10.62 (s, 1H), 8.22 - 8.02 (m, 8H), 7.87 - 7.82 (m, 1H), 7.67 -7.64 (m, 3H).

### Example 23

### 5-chloro-N-[4-(5-phenyl-1,3,4-oxadiazol-2-yl)phenyl]pyridine-2-carboxamide

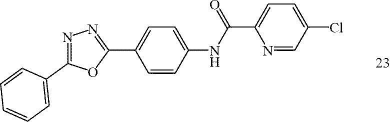 [SMILES: O=C(Nc1ccc(-c2nnc(-c3ccccc3)o2)cc1)c1ccc(Cl)cn1]

The title compound was prepared in analogy to the procedure described for the preparation of Example 1, by using 5-chloropyridine-2-carbonyl chloride instead of propionyl chloride. The product was purified by preparative HPLC to afford Example 23 as a white solid. MS obsd. (ESI⁺) [(M+H)⁺]: 377.1. ¹H NMR (400 MHz, DMSO-d₆) δ ppm: 10.81 (s, 1H), 8.82 (s, 1H), 8.28 - 8.10 (m, 8H), 7.66 - 7.62 (m, 3H).

### Example 24

### N-(5-phenyl-1,3,4-oxadiazol-2-yl)phenyl]pyridazine-4-carboxamide

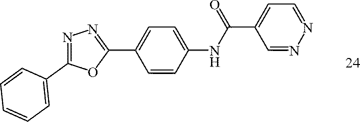 [SMILES: O=C(Nc1ccc(-c2nnc(-c3ccccc3)o2)cc1)c1ccnnc1]

The title compound was prepared in analogy to the procedure described for the preparation of Example 1, by using pyridazine-4-carboxylic acid and propylphosphonic anhydride instead of propionyl chloride. The product was purified by preparative HPLC to afford Example 24 as a white solid. MS obsd. (ESI⁺) [(M+H)⁺]: 344.0. ¹H NMR (400 MHz, DMSO-d₆) δ ppm: 11.05 (s, 1H), 9.68 (dd, J= 1.3, 2.1 Hz, 1H), 9.54 (dd, J= 1.1, 5.3 Hz, 1H), 8.23 - 8.11 (m, 5H), 8.05 (d, J = 8.8 Hz, 2H), 7.70 - 7.60 (m, 3H).

### Example 25

### N-(5-phenyl-1,3,4-oxadiazol-2-yl)phenyl]pyridazine-3-carboxamide

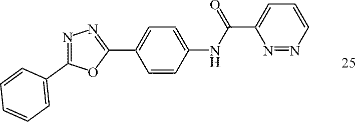 [SMILES: O=C(Nc1ccc(-c2nnc(-c3ccccc3)o2)cc1)c1cccnn1]

The title compound was prepared in analogy to the procedure described for the preparation of Example 1, by using 3-pyridazine carboxylic acid and propylphosphonic anhydride instead of propionyl chloride. The product was purified by preparative HPLC to afford Example 25 as a white solid. MS obsd. (ESI⁺) [(M+H)⁺]: 344.0. ¹H NMR (400 MHz, DMSO-d₆) δ ppm: 11.46 (s, 1H), 9.51 (dd, J= 1.5, 5.0 Hz, 1H), 8.37 (dd, J= 1.4, 8.5 Hz, 1H), 8.28 - 8.22 (m, 2H), 8.20 -8.13 (m, 4H), 8.02 (dd, J= 5.0, 8.5 Hz, 1H), 7.70 - 7.62 (m, 3H).

### Example 26

### N-(5-phenyl-1,3,4-oxadiazol-2-yl)phenyl]oxazole-2-carboxamide

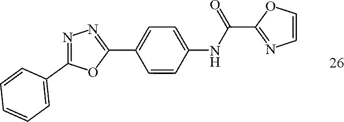 [SMILES: O=C(Nc1ccc(-c2nnc(-c3ccccc3)o2)cc1)c1ncco1]

The title compound was prepared in analogy to the procedure described for the preparation of Example 1, by using oxazole-2-carboxylic acid and O-(7-azabenzotriazol-1-yl)-N,N,N′,N′-tetramethyluronium hexafluorophosphate instead of propionyl chloride. The product was purified by preparative HPLC to afford Example 26 as a light yellow solid. MS obsd. (ESI⁺) [(M+H)⁺]: 333.0. ¹H NMR (400 MHz, DMSO-d₆) δ ppm: 11.22 (br s, 1H), 8.48 - 8.43 (m, 1H), 8.21 - 8.07 (m, 6H), 7.70 - 7.58 (m, 4H).

### Example 27

### N-(5-phenyl-1,3,4-oxadiazol-2-yl)phenyl]thiazole-2-carboxamide

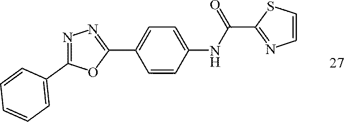 [SMILES: O=C(Nc1ccc(-c2nnc(-c3ccccc3)o2)cc1)c1nccs1]

The title compound was prepared in analogy to the procedure described for the preparation of Example 1, by using thiazole-2-carboxylic acid and O-(7-azabenzotriazol-1-yl)-N,N,N′,N′-tetramethyluronium hexafluorophosphate instead of propionyl chloride. The product was purified by preparative HPLC to afford Example 27 as a light yellow solid. MS obsd. (ESI⁺) [(M+H)⁺]: 349.0. ¹H NMR (400 MHz, DMSO-d₆) δ ppm: 11.18 (s, 1H), 8.21 - 8.11 (m, 8H), 7.69 - 7.61 (m, 3H).

### Example 28

### 2,2-dimethyl-N-[4-(5-phenyl-1,3,4-oxadiazol-2-yl)phenyl]propanamide

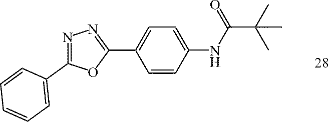 [SMILES: CC(C)(C)C(=O)Nc1ccc(-c2nnc(-c3ccccc3)o2)cc1]

The title compound was prepared in analogy to the procedure described for the preparation of Example 1, by using 2,2-dimethylpropanoyl chloride instead of propionyl chloride. The product was purified by preparative HPLC to afford Example 28 as a white solid. MS obsd. (ESI⁺) [(M+H)⁺]: 322.2. ¹H NMR (400 MHz, DMSO-d₆) δ ppm: 9.56 (s, 1H), 8.15 - 8.06 (m, 4H), 7.95 (d, J = 8.2 Hz, 2H), 7.68 - 7.61 (m, 3H), 1.26 (s, 9H).

### Example 29

### 3,3-dimethyl-N-[4-(5-phenyl-1,3,4-oxadiazol-2-yl)phenyl]butanamide

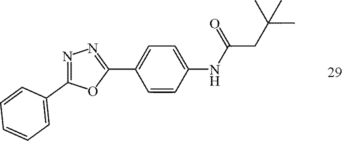 [SMILES: CC(C)(C)CC(=O)Nc1ccc(-c2nnc(-c3ccccc3)o2)cc1]

The title compound was prepared in analogy to the procedure described for the preparation of Example 1, by using 3,3-dimethylbutanoyl chloride instead of propionyl chloride. The product was purified by preparative HPLC to afford Example 29 as a light yellow solid. MS obsd. (ESI⁺) [(M+H)⁺]: 336.3. ¹H NMR (400 MHz, DMSO-d₆) δ ppm: 10.18 (s, 1H), 8.13 (dd, J = 2.1, 7.4 Hz, 2H), 8.07 (d, J = 8.7 Hz, 2H), 7.86 (d, J = 8.8 Hz, 2H), 7.73 - 7.55 (m, 3H), 2.25 (s, 2H), 1.05 (s, 9H).

### Example 30

### 2-Methyl-4-(5-phenyl-1,3,4-oxadiazol-2-yl)aniline

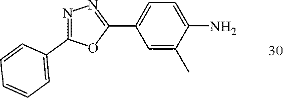 [SMILES: Cc1cc(-c2nnc(-c3ccccc3)o2)ccc1N]

The title compound was prepared according to the following scheme:

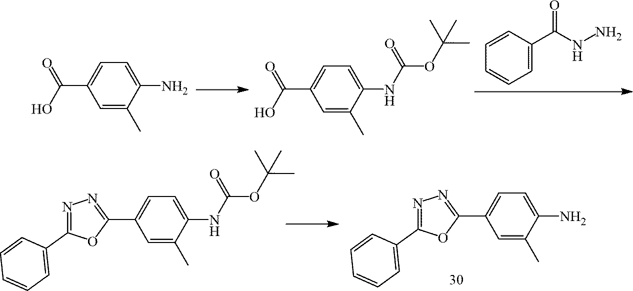

### Step 1: Preparation of 4-(tert-butoxycarbonylamino)-3-methyl-benzoic Acid

To a mixture of 4-amino-3-methylbenzoic acid (500.0 mg, 3.3 mmol) in 1,4-dioxane (10 mL) was added a solution of sodium hydroxide (291.06 mg, 7.3 mmol) in water (10 mL), then di-t-butyldicarbonate (866.3 mg, 4 mmol) was added. After being stirred at 25° C. for 12 h, the reaction was adjusted to pH 5-6 with 1N of HCl. The resulting mixture was filtered. The filter cake was washed with acetonitrile and collected to give 4-(tert-butoxycarbonylamino)-3-methylbenzoic acid (365 mg) as a white solid.

### Step 2: Preparation of Tert-butyl N-[2-methyl-4-(5-phenyl-1,3,4-oxadiazol-2-yl)phenyl]carbamate

A mixture of 4-(tert-butoxycarbonylamino)-3-methyl-benzoic acid (365 mg, 0.7 mmol), benzohydrazide (198 mg, 0.7 mmol), O-(7-azabenzotriazol-1-yl)-N,N,N′,N′-tetramethyluronium hexafluorophosphate (607 mg, 1.6 mmol) and N,N-diisopropylethylamine (536 mg, 4.4 mmol) in acetonitrile (5 mL) was stirred at 25° C. for 12 h. After p-toluenesulfonyl chloride (830 mg, 4.4 mmol) was added, the mixture was stirred for another 3 h. The resulting mixture was treated with ammonium hydroxide solution (3 mL) and filtered. The filter cake was washed with acetonitrile and collected to give tert-butyl N-[2-methyl-4-(5-phenyl-1,3,4-oxadiazol-2-yl)phenyl]carbamate (270 mg) as a white solid.

### Step 3: Preparation of 2-methyl-4-(5-phenyl-1,3,4-oxadiazol-2-yl)aniline (Example 30)

To a solution of tert-butyl N-[2-methyl-4-(5-phenyl-1,3,4-oxadiazol-2-yl)phenyl]carbamate (270.0 mg, 0.8 mmol) in 1,4-dioxane (10 mL) was added HCl in dioxane (10.0 mL,4 N). After being stirred at 25° C. for 12 h, the mixture was concentrated and basified with ammonium hydroxide solution. The residue was purified by preparative HPLC to give 2-methyl-4-(5-phenyl-1,3,4-oxadiazol-2-yl)aniline (Example 30, 128.1 mg) as a white solid. MS obsd. (ESI⁺) [(M+H)⁺]: 252.0. ¹H NMR (400 MHz, DMSO-d₆) δ ppm: 8.11 - 8.05 (m, 2H), 7.72 - 7.65 (m, 2H), 7.64 - 7.58 (m, 3H), 6.74 (d, J = 8.3 Hz, 1H), 5.72 (s, 2H), 2.15 (s, 3H).

### Example 31

### 2-Methoxy-4-(5-phenyl-1,3,4-oxadiazol-2-yl)aniline

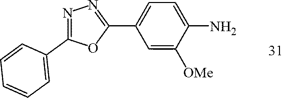 [SMILES: COc1cc(-c2nnc(-c3ccccc3)o2)ccc1N]

The title compound was prepared in analogy to the procedure described for the preparation of Example 30, by using 4-amino-3-methoxybenzoic acid instead of 4-amino-3-methylbenzoic acid. The product was purified by preparative HPLC to afford Example 31 as a white solid. MS obsd. (ESI⁺) [(M+H)⁺]: 268.0. H NMR (400 MHz, DMSO-d6) δ ppm: 8.06 - 8.16 (m, 2H), 7.62 (br d, J= 5.0 Hz, 3H), 7.51 (d, J= 7.7 Hz, 1H), 7.45 (s, 1H), 6.78 (d, J = 8.2 Hz, 1H), 5.63 (s, 2H), 3.90 (s, 3H).

### Example 32

### 2-Fluoro-4-(5-phenyl-1,3,4-oxadiazol-2-yl)aniline

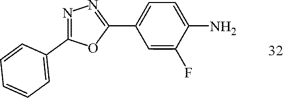 [SMILES: Nc1ccc(-c2nnc(-c3ccccc3)o2)cc1F]

The title compound was prepared in analogy to the procedure described for the preparation of Example 30, by using 4-amino-3-fluorobenzoic acid instead of 4-amino-3-methylbenzoic acid. The product was purified by preparative HPLC to afford Example 32 as a white solid. MS obsd. (ESI⁺) [(M+H)⁺]: 256.0. ¹H NMR (400 MHz, DMSO-d₆) δ ppm: 8.11 (d, J = 6.0 Hz, 2H), 7.59 -7.75 (m, 5H), 6.92 (t, J = 8.7 Hz, 1H), 6.06 (s, 2H).

### Example 33

### N,N-dimethyl-4-(5-phenyl-1,3,4-oxadiazol-2-yl)aniline

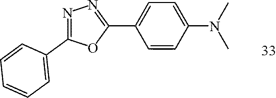 [SMILES: CN(C)c1ccc(-c2nnc(-c3ccccc3)o2)cc1]

The title compound was prepared according to the following scheme:

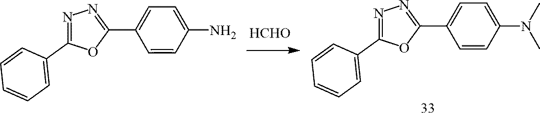

To a mixture of 4-(5-phenyl-1,3,4-oxadiazol-2-yl)aniline (V, 250.0 mg, 1.1 mmol) and formaldehyde (189.8 mg, 6.3 mmol) in methanol (15 mL) was added sodium cyanoborohydride (198.6 mg, 3.2 mmol) at 25° C. After being stirred at 25° C. for 12 h, water was added and extracted with dichloromethane. The organic layer was dried over anhydrous sodium sulfate. The solvent was evaporated under reduced pressure, and the obtained residue was purified by preparative HPLC to give N,N-dimethyl-4-(5-phenyl-1,3,4-oxadiazol-2-yl)aniline (Example 33, 17.02 mg) as a white solid. MS obsd. (ESI⁺) [(M+H)⁺]: 266.0. ¹H NMR (400 MHz, DMSO-d₆) δ ppm: 8.10 (d, J= 6.3 Hz, 2H), 7.92 (d, J= 8.7 Hz, 2H), 7.59 - 7.67 (m, 3H), 6.87 (d, J= 8.7 Hz, 2H), 3.04 (s, 6H).

### BIOLOGICAL EXAMPLES

### Example 34

### PHH Natural Infection Assay

Detailed procedures regarding primary human hepatocyte (PHH) HBV natural infection assay are described as below. One tube of frozen PHH (10 million cells) is thawed in 37° C. water bath and then transferred to 20 mL of PHH thawing medium (Sigma, InVitroGRO HT Medium, Cat. S03319) with gently mixing. The cells were then centrifuged at 80 g/min for 5 min, the supernatant was discarded and the tube was refilled with 25 mL of PHH plating medium (Sigma, InVitroGRO CP Medium, Cat. S03317). The tube was shaken very gently to re-suspend all cells, and then 50 µl of cells were transferred to each well 384-well collagen I coated plate with appropriate liquid handling equipment, e.g. Integra VIAFLO384 or Agilent Bravo. The cells were then cultured for 24 hours in a cell incubator. For HBV infection, after PHH attachment on the culture plate, the plating medium was removed and replenished with PHH culture medium containing HBV virus. The PHH culture medium was prepared with Dulbecco’s Modified Eagle Medium (DMEM)/F12 (1: 1 in volume ratio) containing 10% fetal bovine serum (Gibco, Cat.10099141), 5 ng/mL human epidermal growth factor (Gibco, Cat.PHG0311L), 20 ng/mL dexamethasone (Sigma, Cat.D4902-100 mg), 250 ng/mL human recombinant insulin (Gibco, Cat.41400045) and 100 U/mL penicillin. HBV virus at 200 genome equivalent (GE) per cell with 4% PEG8000 (Sigma, Cat.P1458) containing culture medium were added to the PHH culture medium for infection. The cells were then cultured for 24 hours in cell incubator. Then the cell culture supernatant was removed. The HBV-infected PHH were cultured with sandwich culture method with PHH culture medium containing 1% DMSO and 0.25 mg/mL matrix gel for 72 hours. The supernatant was then refreshed with PHH culture medium containing different concentrations of testing compounds for two times with 72-hour interval. At the end of treatment, the supernatant was collected for viral markers measurements, including HBsAg, HBeAg, HBV DNA and cytotoxicity. HBsAg and HBeAg were detected using alphalisa method using their specific antibodies. For HBV DNA detection, HBV DNA Quantitative Fluorescence Diagnostic Kit (Sansure Biotech Inc.) was used following the manufacture’s protocol. Cytotoxicity was determined using Cell Counting Kit-8 (CCK8, Dojindo Molecular Technologies, Inc.).

The compounds of the present invention were tested for their capacity to inhibit HBsAg and HBeAg as described herein. The Examples were tested in the above assay and found to have IC₅₀ below 10 µM. Results of PHH assay are given in Table 1.

TABLE 1

Activity data of compounds of this invention

| Example No. | HBsAg IC₅₀ (µM) | HBeAg IC₅₀ (µM) | CC₅₀ (µM) |
|---|---|---|---|
| Example 1 | 1.19 | 4.85 | >100 |
| Example 2 | 3.00 | 43.10 | >100 |
| Example 3 | 2.39 | 3.01 | >100 |
| Example 4 | 2.80 | 3.43 | >100 |
| Example 5 | 0.59 | 0.69 | >100 |
| Example 6 | 1.33 | 2.20 | >100 |
| Example 7 | 0.51 | 0.77 | >100 |
| Example 8 | 4.17 | 5.06 | >100 |
| Example 9 | 8.23 | 1.69 | >100 |
| Example 10 | 1.61 | 2.17 | >100 |
| Example 11 | 6.40 | 12.65 | >100 |
| Example 12 | 6.20 | 6.33 | >100 |
| Example 13 | 5.62 | 4.43 | >100 |
| Example 14 | 3.59 | 2.88 | >100 |
| Example 15 | 1.94 | 1.94 | >100 |
| Example 16 | 1.35 | 2.91 | >100 |
| Example 17 | 1.46 | 2.79 | >100 |
| Example 18 | 2.54 | 2.26 | >100 |
| Example 19 | 4.80 | 2.80 | >100 |
| Example 20 | 1.39 | 2.56 | >100 |
| Example 21 | 2.00 | 1.91 | >100 |
| Example 22 | 0.96 | 1.59 | >100 |
| Example 23 | 1.23 | 2.12 | >100 |
| Example 24 | 1.20 | 1.60 | >100 |
| Example 25 | 1.09 | 1.27 | >100 |
| Example 26 | 0.91 | 1.42 | >100 |
| Example 27 | 1.52 | 1.71 | >100 |
| Example 28 | 7.65 | 4.23 | >100 |
| Example 29 | 5.10 | 9.57 | >100 |
| Example 30 | 1.43 | 3.21 | >100 |
| Example 31 | 1.49 | 4.17 | >100 |
| Example 32 | 2.39 | 2.81 | >100 |
| Example 33 | 7.69 | 91.65 | >100 |

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

It is to be understood that the invention is not limited to the particular embodiments and aspects of the disclosure described above, as variations of the particular embodiments and aspects may be made and still fall within the scope of the appended claims. All documents cited to or relied upon herein are expressly incorporated by reference.

1. A compound of the formula (I), 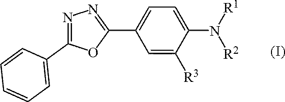
wherein
R¹ is H, C₁₋₆alkyl or —C(O)—R⁴; wherein
R⁴ is C₁₋₆alkyl, C₁₋₆alkoxyC₁₋₆alkyl, C₃₋₇cycloalkyl, phenyl or heterocyclyl; wherein phenyl and heterocyclyl are unsubstituted or substituted by one or two or three substituents independently selected from halogen, C₁₋₆alkyl, C₁₋₆alkoxy, haloC₁₋ ₆alkoxy and CN;
R² is H or C₁₋₆alkyl;
R³ is H, halogen, C₁₋₆alkyl or C₁₋₆alkoxy;
wherein with the proviso that R¹, R² and R³ are not H simultaneously;
or a pharmaceutically acceptable salt thereof.

2. A compound according to claim 1, wherein
R¹ is H, C₁₋₆alkyl or —C(O)—R⁴; wherein
R⁴ is C₁₋₆alkyl, C₁₋₆alkoxyC₁₋₆alkyl, C₃₋₇cycloalkyl, phenyl, pyridyl, pyridazinyl, oxazolyl or thiazolyl; wherein phenyl and pyridyl are unsubstituted or substituted by one or two or three substituents independently selected from halogen, C₁₋₆alkyl, C₁₋₆alkoxy, halogenC₁₋₆alkoxy and CN;
R² is H or C₁₋₆alkyl;
R³ is H, halogen, C₁₋₆alkyl or C₁₋₆alkoxy;
wherein with the proviso that R¹, R² and R³ are not H simultaneously;
or a pharmaceutically acceptable salt thereof.

3. A compound according to claim 2, wherein
R¹ is H, methyl or —C(O)—R⁴; wherein
R⁴ is ethyl, propyl, isopropyl, butyl, isobutyl, tert-butyl, 2,2-dimethylpropyl, methoxymethyl, methoxyethyl, cyclopentyl, phenyl, pyridyl, pyridazinyl, oxazolyl or thiazolyl; wherein phenyl and pyridyl are unsubstituted or substituted by one or two or three substituents independently selected from F, Cl, methyl, methoxy, difluoromethoxy, trifluoromethoxy and CN;
R² is H or methyl;
R³ is H, F, methyl or methoxy;
wherein with the proviso that R¹, R² and R³ are not H simultaneously;
or a pharmaceutically acceptable salt thereof.

4. A compound according to claim 1, or a pharmaceutically acceptable salt thereof, wherein R¹ is C(O)-R⁴; wherein R⁴ is C₁₋₆alkyl, C₁₋₆alkoxyC₁₋₆alkyl, C₃₋₇cycloalkyl, pyridyl, pyridazinyl or oxazolyl; wherein pyridyl is substituted one time by halogen.

5. A compound according to claim 4, or a pharmaceutically acceptable salt thereof, wherein R⁴ is ethyl, isopropyl, methoxymethyl, cyclopentyl, pyridyl, pyridazinyl or oxazolyl; wherein pyridyl is substituted one time by Cl.

6. A compound according to claim 1, or a pharmaceutically acceptable salt thereof, wherein R² is H; and R³ is H.

7. A compound according to claim 1 having the formula (II), 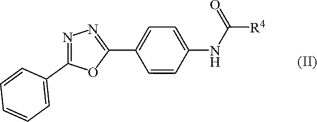
wherein R⁴ is C₁₋₆alkyl, C₁₋₆alkoxyC₁₋₆alkyl, C₃₋₇cycloalkyl, pyridyl, pyridazinyl or oxazolyl;
wherein pyridyl is substituted one time by halogen, or a pharmaceutically acceptable salt thereof.

8. A compound according to claim 7, or a pharmaceutically acceptable salt thereof, wherein R⁴ is ethyl, isopropyl, methoxymethyl, cyclopentyl, pyridyl, pyridazinyl or oxazolyl; wherein pyridyl is substituted one time by Cl.

9. A compound according to claim 1, selected from the group consisting of:
N-[4-(5-phenyl-1,3,4-oxadiazol-2-yl)phenyl]propanamide;
N-[4-(5-phenyl-1,3,4-oxadiazol-2-yl)phenyl]butanamide;
N-[4-(5-phenyl-1,3,4-oxadiazol-2-yl)phenyl]pentanamide;
3-methyl-N-[4-(5-phenyl-1,3,4-oxadiazol-2-yl)phenyl]butanamide;
3-methoxy-N-[4-(5-phenyl-1,3,4-oxadiazol-2-yl)phenyl]propanamide;
2-methyl-N-[4-(5-phenyl-1,3,4-oxadiazol-2-yl)phenyl]propanamide;
N-[4-(5-phenyl-1,3,4-oxadiazol-2-yl)phenyl]cyclopentanecarboxamide;
2-fluoro-N-[4-(5-phenyl-1,3,4-oxadiazol-2-yl)phenyl]benzamide;
2-chloro-N-[4-(5-phenyl-1,3,4-oxadiazol-2-yl)phenyl]benzamide;
3-fluoro-5-methyl-N-[4-(5-phenyl-1,3,4-oxadiazol-2-yl)phenyl]benzamide;
3-chloro-5-methyl-N-[4-(5-phenyl-1,3,4-oxadiazol-2-yl)phenyl]benzamide;
4-fluoro-N-[4-(5-phenyl-1,3,4-oxadiazol-2-yl)phenyl]benzamide;
4-chloro-N-[4-(5-phenyl-1,3,4-oxadiazol-2-yl)phenyl]benzamide;
N-[4-(5-phenyl-1,3,4-oxadiazol-2-yl)phenyl]-2-(trifluoromethoxy)benzamide;
2-(difluoromethoxy)-N-[4-(5-phenyl-1,3,4-oxadiazol-2-yl)phenyl]benzamide;
N-[4-(5-phenyl-1,3,4-oxadiazol-2-yl)phenyl]-3-(trifluoromethoxy)benzamide;
3-(difluoromethoxy)-N-[4-(5-phenyl-1,3,4-oxadiazol-2-yl)phenyl]benzamide;
4-cyano-N-[4-(5-phenyl-1,3,4-oxadiazol-2-yl)phenyl]benzamide;
N-[4-(5-phenyl-1,3,4-oxadiazol-2-yl)phenyl]pyridine-4-carboxamide;
2-chloro-N-[4-(5-phenyl-1,3,4-oxadiazol-2-yl)phenyl]pyridine-4-carboxamide;
2-methoxy-N-[4-(5-phenyl-1,3,4-oxadiazol-2-yl)phenyl]pyridine-4-carboxamide;
6-chloro-N-[4-(5-phenyl-1,3,4-oxadiazol-2-yl)phenyl]pyridine-2-carboxamide;
5-chloro-N-[4-(5-phenyl-1,3,4-oxadiazol-2-yl)phenyl]pyridine-2-carboxamide;
N-[4-(5-phenyl-1,3,4-oxadiazol-2-yl)phenyl]pyridazine-4-carboxamide;
N-[4-(5-phenyl-1,3,4-oxadiazol-2-yl)phenyl]pyridazine-3-carboxamide;
N-[4-(5-phenyl-1,3,4-oxadiazol-2-yl)phenyl]oxazole-2-carboxamide;
N-[4-(5-phenyl-1,3,4-oxadiazol-2-yl)phenyl]thiazole-2-carboxamide;
2,2-dimethyl-N-[4-(5-phenyl-1,3,4-oxadiazol-2-yl)phenyl]propanamide;
3,3-dimethyl-N-[4-(5-phenyl-1,3,4-oxadiazol-2-yl)phenyl]butanamide;
2-methyl-4-(5-phenyl-1,3,4-oxadiazol-2-yl)aniline;
2-methoxy-4-(5-phenyl-1,3,4-oxadiazol-2-yl)aniline;
2-fluoro-4-(5-phenyl-1,3,4-oxadiazol-2-yl)aniline; and,
N,N-dimethyl-4-(5-phenyl-1,3,4-oxadiazol-2-yl)aniline;
or a pharmaceutically acceptable salt thereof.

10. A compound according to claim 1 selected from the group consisting of:
N-[4-(5-phenyl-1,3,4-oxadiazol-2-yl)phenyl]propanamide;
3-methoxy-N-[4-(5-phenyl-1,3,4-oxadiazol-2-yl)phenyl]propanamide;
2-methyl-N-[4-(5-phenyl-1,3,4-oxadiazol-2-yl)phenyl]propanamide;
N-[4-(5-phenyl-1,3,4-oxadiazol-2-yl)phenyl]cyclopentanecarboxamide;
6-chloro-N-[4-(5-phenyl-1,3,4-oxadiazol-2-yl)phenyl]pyridine-2-carboxamide;
N-[4-(5-phenyl-1,3,4-oxadiazol-2-yl)phenyl]pyridazine-3-carboxamide; and,
N-[4-(5-phenyl-1,3,4-oxadiazol-2-yl)phenyl]oxazole-2-carboxamide;
or a pharmaceutically acceptable salt thereof.

11. A process for the preparation of a compound according to claim 1 comprising at least one of the following steps,
(a) coupling of a compound of formula (II), 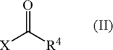
with 4-(5-phenyl-1,3,4-oxadiazol-2-yl)aniline in the presence of a coupling reagent and a base;
(b) deprotection of a compound of formula (V), 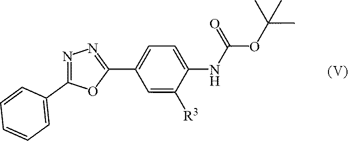
in the presence of an acid;
(c) reductive amination of a compound of formula (I-2), 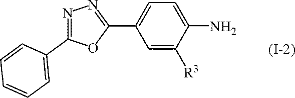
with formaldehyde in the presence of sodium cyanoborohydride;
wherein R¹ to R⁴ are defined as any one of claims 1 to 3; X is halogen or OH.

12. A compound according to claim 1 for use as therapeutically active substance.

13. A pharmaceutical composition comprising a compound in accordance with claim 1 and a therapeutically inert carrier.

14. The use of a compound according to claim 1 for the treatment of HBV infection.

15. The use of a compound according to claim 1 for the preparation of a medicament for the treatment of HBV infection.

16. The use of a compound according to claim 1 for the inhibition of HBeAg.

17. The use of a compound according to claim 1 for the inhibition of HBsAg.

18. The use of a compound according to claim 1 for the inhibition of HBV DNA.

19. A compound according to claim 1 for use in the treatment of HBV infection.

20. A compound according to claim 1 when manufactured according to a process of claim 11.

21. A method for the treatment of HBV infection, which method comprises administering an effective amount of a compound as defined in claim 1.
